(19) United States
(12) Patent Application Publication
(10) Pub. No.: US 2023/0067978 A1
(43) Pub. Date: Mar. 2, 2023

(54) Small-size Multi-axis Ankle Prosthesis

(71) Applicant: Jilin University, Changchun City (CN)

(72) Inventors: Kunyang WANG, Changchun City (CN); Lei REN, Changchun City (CN); Zhihui QIAN, Changchun City (CN); Xuewei LU, Changchun City (CN); Wei LIANG, Changchun City (CN); Yi ZENG, Changchun City (CN); Luquan REN, Changchun City (CN)

(21) Appl. No.: 17/896,054

(22) Filed: Aug. 25, 2022

(30) Foreign Application Priority Data
Aug. 27, 2021 (CN) 202110992322.9

(51) Int. Cl. A61F 2/66

(52) U.S. Cl. CPC A61F 2/6607; A61F 2002/5075

(57) ABSTRACT

A small-size multi-axis ankle joint prosthesis is provided. Two ends of each of four springs are fixedly connected with two spring seats at an upper part and at the lower part respectively. The joint axes are positioned in a space surrounded by the lower part, the four springs and the upper part. Front and rear perforated blocks in the joint axes are respectively in bolted connection with a fifth arc hole pair g and a sixth arc hole pair h of a second arc plate in the upper part. Left and right perforated blocks in the joint axes are respectively in bolted connection with a second arc hole pair e and a third arc hole pair f of the first arc plate in the lower part.

### CROSS-REFERENCE TO RELATED APPLICATION(S)

The present disclosure claims priority to Chinese Patent Application No. 202110992322.9, filed on Aug. 27, 2021 and entitled “Small-Size Multi-Axis Ankle Prosthesis”, the contents of which are hereby incorporated by reference in its entirety.

### TECHNICAL FIELD

The present disclosure belongs to the technical field of bionic human prosthesis, particularly to a small-size multi-axis ankle prosthesis.

### BACKGROUND ART

Ankles of human bodies can be understood as multi-axis structures, including talocrural and subtalar joint axes. Joint axis angles of different individuals are different. Human ankles are multi-axis structures, and thus can act smoothly. At present, most ankle prostheses in the market adopt a single axis fixation design or two axes fixation design, and has kinematic mechanical properties quite different from those of healthy people, causing a wearer to suffer from unnatural gait, uncomfortable walking and low adaptability to different environments and roads during use.

### SUMMARY

An object of some embodiments of the present disclosure is to provide a small-size multi-axis ankle prosthesis product with biomechanical characteristics of human ankle joint and multi-axis structure, which can adjust joint axis angles individually according to different patients, so as to improve movement coordination between the affected side limb and the normal side limb of wearers and improve environmental adaptability and wearing comfort of the ankle prosthesis, thereby coping with different situations and terrains, and which is simple in structure, small in volume, clear in an adjustment process and stable in structure, thereby effectively reducing energy consumption of the wearers.

A small-size multi-axis ankle prosthesis provided by the present disclosure, includes a lower part A, a joint axis B, an upper part C, a first spring 1, a second spring 2, a third spring 3 and a fourth spring 4. The lower part A includes a base plate 5, a first ball cover group 6, a first ball rod group 7, a first spring seat group 8 and a first arc plate assembly 9. The base plate 5 is disc-shaped, and an outer periphery of the base plate is uniformly provided with four flanges of a first flange group.

The first arc plate assembly 9 includes a first arc plate 15 and a transverse plate 14, and the first arc plate 15 is fixedly connected on an upper surface of the transverse plate 14; two arc holes of a second arc hole pair e are provided on the first arc plate 15 adjacent to a front end thereof; two arc holes of a third arc hole pair f are provided on the first arc plate 15 adjacent to a rear end thereof.

The joint axis B includes a subtalar joint axis 16, a left connecting block 17a, a right connecting block 17b, an ankle axis 18, a front connecting block 19a, a rear connecting block 19b, a bolt 20, and an oil-free bushing group 21. The left connecting block 17a includes a first cylindrical cap 24a and a first perforated block 25a which is fixedly connected to a center portion on a front end of the first cylindrical cap 24a. The right connecting block 17b includes a second cylindrical cap 24b and a second perforated block 25b which is fixedly connected to a center portion on a rear end of the second cylindrical cap 24b.

The front connecting block 19a includes a third cylindrical cap 28a and a third perforated block 29a which is fixedly connected to a center portion on a left end of the third cylindrical cap 28a; the rear connecting block 19b includes a fourth cylindrical cap 28b and a fourth perforated block 29b which is fixedly connected to a center portion on a right end of the fourth cylindrical cap 28b.

The upper part C includes a second arc plate assembly 30, a second spring seat group 31, a second ball rod group 32, a second ball cover group 33 and a top plate 34, the top plate 34 is disc-shaped and an outer periphery of the top plate is uniformly provided with four flanges of a second flange group 37.

The second arc plate assembly 30 includes a second arc plate 35 and a transverse plate 36; the second arc plate 35 is fixedly connected to a bottom of the transverse plate 36, a fifth arc hole pair g is provided on the second arc plate 35 adjacent to a left lower end thereof, and a sixth arc hole pair h is provided on the second arc plate 35 adjacent to a right lower end thereof.

The four flanges of the second flange group 37 of the top plate 34 in the upper part C are symmetrical with the four flanges of the first flange group 10 of the base plate 5 in the lower part A, in an up-down direction. Upper ends of the first spring 1, the second spring 2, the third spring 3 and the fourth spring 4 are respectively fixedly connected with four spring seats of the second spring seat group 31 in the upper part C. Lower ends of the first spring 1, the second spring 2, the third spring 3 and the fourth spring 4 are respectively fixedly connected with four spring seats of the first spring seat group 8 in the lower part A. The joint axis B is located in a space surrounded by the lower part A, the first spring 1, the second spring 2, the third spring 3, the fourth spring 4 and the upper part C. The third perforated block 29a of the front connecting block 19a in the joint axis B is connected with the fifth arc hole pair g of the second arc plate 35 in the upper part C through a bolt. The fourth perforated block 29b of the rear connecting block 19b in the joint axis B is connected with the sixth arc hole pair h of the second arc plate 35 in the upper part C through a bolt. The first perforated block 25a of the left connecting block 17a in the joint axis B is connected with the second arc hole pair e of the first arc plate 15 in the lower part A through a bolt. The second perforated block 25b of the right connecting block 17b in the joint axis B is connected with the third arc hole pair f of the first arc plate 15 in the lower part A through a bolt.

In some embodiments, the four flanges of the first flange group 10 of the base plate 5 in the lower part A are provided with four holes of the first stepped hole group a respectively, a back surface of the base plate 5 is provided with a boss 11, and a center portion of the base plate 5 is provided with a first threaded hole c, two arc holes of the first arc hole pair b provided in the base plate 5 are symmetrically located on a front side and a rear side of the boss 11 respectively. The first ball cover group 6 includes four ball covers; the first ball rod group 7 includes four ball rods, each of which is formed by fixedly connecting a first ball body 12 and a first threaded rod 13; the first spring seat group 8 includes four spring seats, and a center of each spring seat is provided with a second threaded hole d. Two ends of the transverse plate 14 are connected with the two arc holes of the first arc hole pair b in the base plate 5 through bolts; the four first ball bodies 12 of the first ball rod group 7 are movably connected with four stepped holes of the first stepped hole group a in the base plate 5. Four ball covers of the first ball cover group 6 are respectively fixedly connected under the four flanges of the first flange group 10; the four second threaded holes d of the first spring seat group 8 are in threaded connection with the four first threaded rods 13 of the first ball rod group 7.

In some embodiments, the oil-free bushing group 21 in the joint axis B includes four oil-free bushings. The subtalar joint axis 16 includes a first round plate 22 with a threaded hole, and a first cylinder pair 23; two cylinders of the first cylinder pair 23 are respectively fixedly connected to a front side and a rear side of the first round plate 22 with the threaded hole. The ankle axis 18 includes a second round plate 26 with a threaded hole, and a second cylinder pair 27; and two cylinders of the second cylinder pair 27 are fixedly connected to a left side and a right side of the second round plate 26 with the threaded hole respectively. The ankle axis 18 and the subtalar joint axis 16 are arranged with one on another and in a cross mode, and are fixedly connected by the bolt 20; the oil-free bushing group 21 includes four oil-free bushings; two connecting blocks of the first connecting block pair 17 are movably connected with the two cylinders of the second cylinder pair 27 in the ankle axis 18 through left and right oil-free bushings of the oil-free bushing group 21; two connecting blocks of the second connecting block pair 19 are movably connected with the two cylinders of the first cylinder pair 23 in the subtalar joint axis 16 through front and rear oil-free bushings of the oil-free bushing group 21.

In some embodiments, the four flanges of the second flange group 37 of the top plate 34 in the upper part C are provided with four stepped holes of the second stepped hole group j respectively. A tetragonal pyramid 38 is fixedly connected on a center of the top plate 34; two arc holes of the fourth arc hole pair i in the top plate 34 are symmetrically located on left and right sides of the tetragonal pyramid 38. The second ball cover group 33 includes four ball covers, which are respectively fixedly connected on the four flanges of the second flange group 37. The second ball rod group 32 includes four ball rods, each of which is formed by fixedly connecting the second ball body 40 and the second threaded rod 39. The second spring seat group 31 includes four spring seats, and a center of each spring seat is provided with a second threaded hole k. Two ends of the transverse plate 36 are connected with the two arc holes of the fourth arc hole pair i of the top plate 34 through bolts; four second ball bodies 40 of the second ball rod group 32 are movably connected with four stepped holes of the second stepped hole group j of the top plate 34. Four second threaded holes k of the second spring seat group 31 are in threaded connection with four second threaded rods 39 of the second ball rod group 32.

The angle adjustment process of the joint axis of some embodiments in the present disclosure is as follows:

1. unscrewing the bolt 20 connecting the subtalar joint axis 16 and the ankle axis 18;

2. unscrewing the bolt connecting the second arc plate assembly 30 and the top plate 34, unscrewing the bolt connecting the fifth arc hole pair g and the third perforated block 29a of the left connecting block 17a, and unscrewing the bolt connecting the sixth arc hole pair h and the fourth perforated block 29b of the right connecting block 17b.

3. rotating the second arc plate assembly 30 to an ideal position along the fourth arc hole pair i, and screwing the bolt connecting the second arc plate assembly 30 and the top plate 34;

4. rotating the ankle axis 18 to an ideal position along the fifth arc hole pair g, screwing the bolt connecting the fifth arc hole pair g and the third perforated block 29a of the left connecting block 17a, and screwing the bolt connecting the sixth arc hole pair h and the fourth perforated block 29b of the right connecting block 17b;

5. unscrewing the bolt connecting the first arc plate assembly 9 and the base plate 5, unscrewing the bolt connecting the second arc hole pair e and the first perforated block 25a of the front connecting block 19a, and unscrewing the bolt connecting the third arc hole pair f and the second perforated block 25b of the rear connecting block 19b;

6. rotating the first arc plate assembly 9 to an ideal position along the first arc hole pair b, and screwing the bolt connecting the first arc plate assembly 9 and the base plate 5;

7. rotating the subtalar joint axis 16 to an ideal position along the second arc hole pair e, screwing the bolt connecting the second arc hole pair e and the first perforated block 25a of the front connecting block 19a, and screwing the bolt connecting the third arc hole pair f and the second perforated block 25b of the rear connecting block 19b; and

8. screwing the bolt 20 connecting the subtalar joint axis 16 and the ankle axis 18.

So far, the angle adjustment of the joint axis is completed.

Some embodiments of the present disclosure have the following beneficial effects.

Some embodiments of the present disclosure can effectively overcome the problems of poor adaptability, low adaptability to road conditions and uncomfortable walking of the ankle prosthesis with a fixed angle, and reduce damages to the residual limb. Wearers can adjust angles of the joint axis of the prosthesis product as desired, to achieve the most comfortable wearing experience. The small-sized structure is closer to the human body, with high matching and high structural stability, so the wearers can use it simply, conveniently, safely and stably.

### BRIEF DESCRIPTION OF THE DRAWINGS

FIG. 1 is a perspective view of a small-size multi-axis ankle prosthesis;

FIG. 2 is a perspective view of a lower part A;

FIG. 3 is a perspective view of a base plate 5;

FIG. 4 is a perspective view of a ball rod of a first ball rod group 7;

FIG. 5 is a perspective view of a spring seat of a first spring seat group 8;

FIG. 6 is a cross-section view showing connection among the first ball rod group 7, a first ball cover group 6 and a first flange group 10;

FIG. 7 is a perspective view of a first arc plate assembly 9;

FIG. 8 is a perspective view of a joint axis B;

FIG. 9 is a perspective view of the joint axis B after removing a front connecting block 19a, a rear connecting block 19b, a left connecting block 17a, and a right connecting block 17b;

FIG. 10 is a perspective view of a subtalar joint axis 16;

FIG. 11 is a perspective view of the front connecting block 19a;

FIG. 12 is a perspective view of the rear connecting block 19b;

FIG. 13 is a perspective view of an ankle axis 18;

FIG. 14 is a perspective view of the left connecting block 17a;

FIG. 15 is a perspective view of the right connecting block 17b;

FIG. 16 is a perspective view of an upper part C;

FIG. 17 is a perspective view of a second arc plate assembly 30;

FIG. 18 is a perspective view of a top plate 34;

FIG. 19 is a perspective view of a ball rod of a second ball rod group 32;

FIG. 20 is a perspective view of a spring seat of a second spring seat group 31; and

FIG. 21 is a cross-section view showing connection among the second ball rod group 32, the second ball cover group 33 and the second flange group 37.

Reference numerals: A—lower part; B—joint axis; C—upper part; 1—first spring; 2—second spring; 3—third spring; 4—fourth spring; 5—base plate; 6—first ball cover group; 7—first ball rod group; 8—first spring seat group; 9—first arc plate assembly; 10—first flange group; 11—boss; 12—first ball body; 13—first threaded rod; 14—transverse plate; 15—first arc plate; 16—subtalar joint axis; 17a—left connecting block; 17b—right connecting block; 18—ankle axis; 19a—front connecting block; 19b—rear connecting block; 20—bolt; 21—oil-free bushing group; 22 first round plate with threaded hole; 23 first cylinder pair; 24a—first cylinder cap; 24b—second cylinder cap; 25a—first perforated block; 25b—second perforated block; 26—second round plate with threaded hole; 27—second cylinder pair; 28a—third cylinder cap; 28b—fourth cylinder cap; 29a—third perforated block; 29b—fourth perforated block; 30—second arc plate assembly; 31—second spring seat group; 32—second ball rod group; 33—second ball cover group; 34—top plate; 35—second arc plate; 36—transverse plate; 37—second flange group; 38—tetragonal pyramid; 39—second threaded rod; 40—second ball body; a—first stepped hole group; b—first arc hole pair; c—first threaded hole; d—second threaded hole; e—second arc hole pair; f—third arc hole pair; g—fifth arc hole pair; h—sixth arc hole pair; i—fourth arc hole pair; j—second stepped hole group; k—second threaded hole.

### DETAILED DESCRIPTION OF THE EMBODIMENTS

The present disclosure is described below in conjunction with the accompanying drawings.

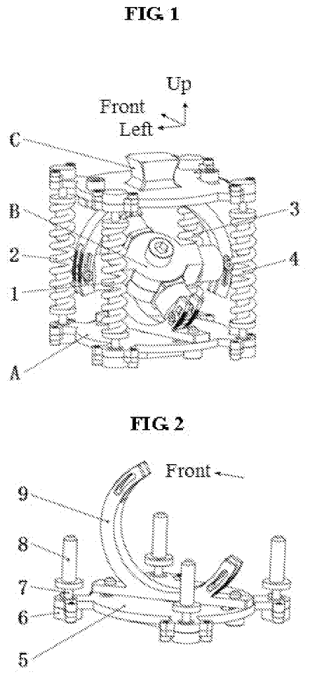

As shown in FIG. 1, a small-size multi-axis ankle prosthesis of the present disclosure includes a lower part A, a joint axis B, an upper part C, a first spring 1, a second spring 2, a third spring 3 and a fourth spring 4. The lower part A includes a base plate 5, a first ball cover group 6, a first ball rod group 7, a first spring seat group 8 and a first arc plate assembly 9. The base plate 5 is disc-shaped, and an outer periphery of the base plate is uniformly provided with four flanges of a first flange group 10.

The first arc plate assembly 9 includes a first arc plate 15 and a transverse plate 14, and the first arc plate 15 is fixedly connected to the transverse plate 14. Two arc holes of a second arc hole pair e are provided on the first arc plate 15 adjacent to a front end thereof; two arc holes of a third arc hole pair f are provided on the first arc plate 15 adjacent to a rear end thereof.

The joint axis B includes a subtalar joint axis 16, a left connecting block 17a, a right connecting block 17b, an ankle axis 18, a front connecting block 19a, a rear connecting block 19b, a bolt 20, and an oil-free bushing group 21. The left connecting block 17a includes a first cylindrical cap 24a and a first perforated block 25a, and the first perforated block 25a is fixedly connected to a center portion on a front end of the first cylindrical cap 24a; the right connecting block 17b includes a second cylindrical cap 24b and a second perforated block 25b, and the second perforated block 25b is fixedly connected to a center portion on a rear end of the second cylindrical cap 24b.

The front connecting block 19a includes a third cylindrical cap 28a and a third perforated block 29a, and the third perforated block 29a is fixedly connected to a center portion on a left end of the third cylindrical cap 28a; the rear connecting block 19b includes a fourth cylindrical cap 28b and a fourth perforated block 29b, which is fixedly connected to a center portion on a right end of the fourth cylindrical cap 28b.

The upper part C includes a second arc plate assembly 30, a second spring seat group 31, a second ball rod group 32, a second ball cover group 33 and a top plate 34. The top plate 34 is disc-shaped, and an outer periphery of the top plate is uniformly provided with four flanges of a second flange group 37.

The second arc plate assembly 30 includes a second arc plate 35 and a transverse plate 36. The second arc plate 35 is fixedly connected to a bottom of the transverse plate 36. A fifth arc hole pair g is provided on the second arc plate 35 adjacent to a left lower end thereof, and a sixth arc hole pair h is provided on the second arc plate 35 adjacent to a right lower end thereof.

The four flanges of the second flange group 37 of the top plate 34 in the upper part C are symmetrical with the four flanges of the first flange group 10 of the base plate 5 in the lower part A, in an up-down direction. Upper ends of the first spring 1, the second spring 2, the third spring 3 and the fourth spring 4 are respectively fixedly connected with the four spring seats of the second spring seat group 31 in the upper part C. Lower ends of the first spring 1, the second spring 2, the third spring 3 and the fourth spring 4 are respectively fixedly connected with the four spring seats of the first spring seat group 8 in the lower part A. The joint axis B is located in a space surrounded by the lower part A, the first spring 1, the second spring 2, the third spring 3, the fourth spring 4 and the upper part C. The third perforated block 29a of the front connecting block 19a in the joint axis B is connected with the fifth arc hole pair g of the second arc plate 35 in the upper part C through bolts. The fourth perforated block 29b of the rear connecting block 19b in the joint axis B is connected with the sixth arc hole pair h of the second arc plate 35 in the upper part C through bolts. The first perforated block 25a of the left connecting block 17a in the joint axis B is connected with the second arc hole pair e of the first arc plate 15 in the lower part A through bolts. The second perforated block 25b of the right connecting block 17b in the joint axis B is connected with the third arc hole pair f of the first arc plate 15 in the lower part A through bolts.

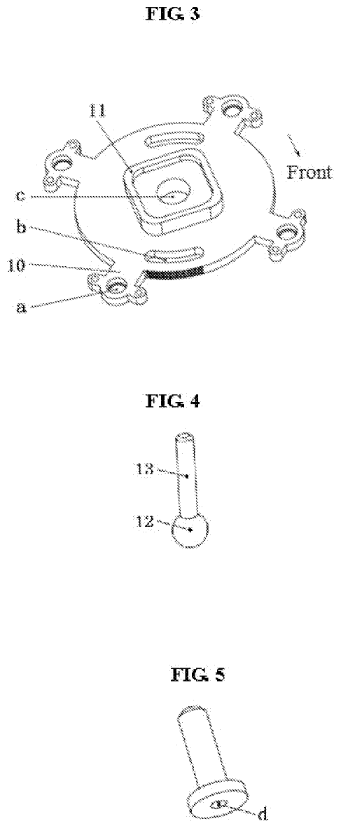

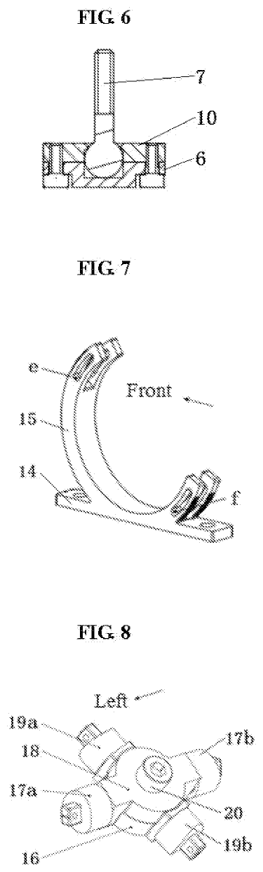

As shown in FIGS. 2 to 7, in some embodiments, the four flanges of the first flange group 10 of the base plate 5 in the lower part A are provided with four holes of the first stepped hole group a. A back surface of the base plate 5 is provided with a boss 11, and a center portion of the base plate 5 is provided with a first threaded hole c. Two arc holes of the first arc hole pair b provided on the base plate 5 are symmetrically located on a front side and a rear side of the boss 11. The first ball cover group 6 includes four ball covers. The first ball rod group 7 includes four ball rods. Each of the ball rods is formed by fixedly connecting a first ball body 12 and a first threaded rod 13. The first spring seat group 8 includes four spring seats, and a center of each spring seat is provided with a second threaded hole d. Two ends of the transverse plate 14 are connected with two arc holes of the first arc hole pair b in the base plate 5 through bolts. The four first ball bodies 12 of the first ball rod group 7 are movably connected with the four stepped holes of the first stepped hole group a in the base plate 5. Four ball covers of the first ball cover group 6 are respectively fixedly connected under the four flanges of the first flange group 10. The four second threaded holes d of the first spring seat group 8 are threaded with the four first threaded rods 13 of the first ball rod group 7.

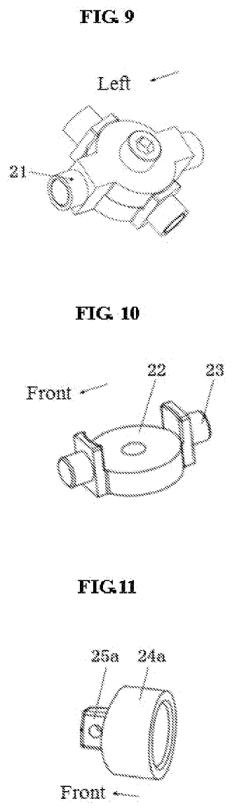

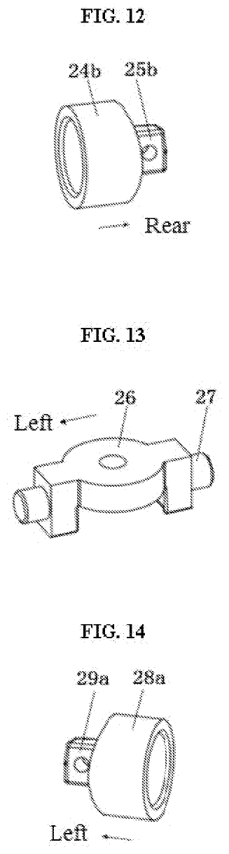

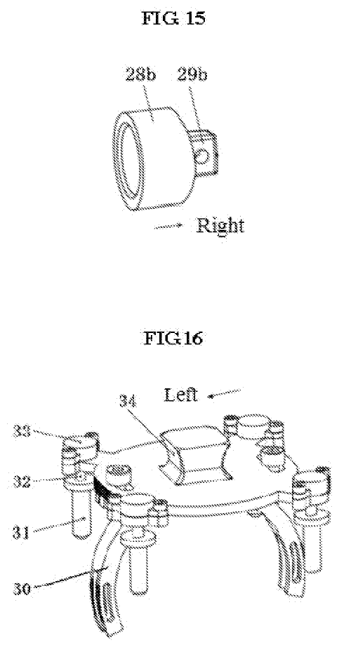

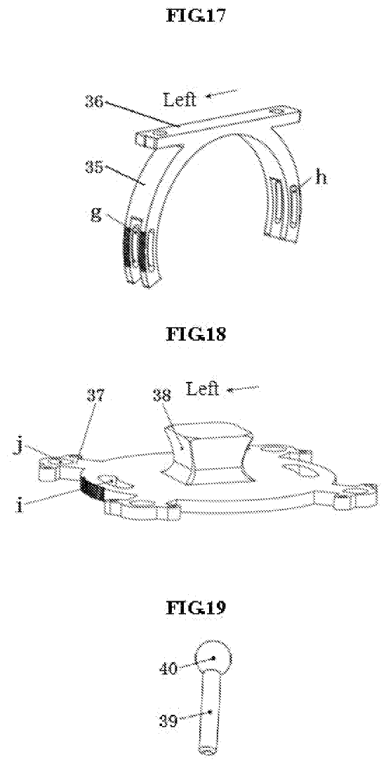

As shown in FIG. 8 to FIG. 15, in some embodiments, the oil-free bushing group 21 in the joint axis B includes four oil-free bushings. Subtalar joint axis 16 includes a first round plate 22 with a threaded hole and a first cylinder pair 23. Two cylinders of the first cylinder pair 23 are respectively fixedly connected to a front side and a rear side of the first round plate 22 with a threaded hole. The ankle axis 18 includes a second round plate 26 with a threaded hole and a second cylinder pair 27, and the two cylinders of the second cylinder pair 27 are fixedly connected to a left side and a right side of the second round plate 26 with a threaded hole respectively. The ankle axis 18 and the subtalar joint axis 16 are arranged with one on the other and in a cross mode, and are fixedly connected by bolts 20. The oil-free bushing group 21 includes four oil-free bushings. Two connecting blocks of the first connecting block pair 17 are movably connected with two cylinders of the second cylinder pair 27 in the ankle axis 18 through the left and right oil-free bushings of the oil-free bushing group 21. Two connecting blocks of the second connecting block pair 19 are movably connected with two cylinders of the first cylinder pair 23 in the subtalar joint axis 16 through the front and rear oil-free bushings of the oil-free bushing group 21.

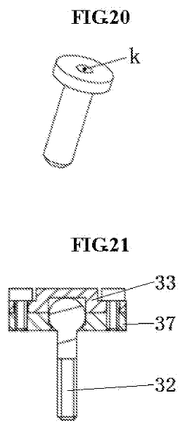

As shown in FIGS. 16 to 21, four flanges of the second flange group 37 of the top plate 34 in the upper part C are provided with four stepped holes of the second stepped hole group j. A tetragonal pyramid 38 is fixedly connected on a center of the top plate 34. Two arc holes of the fourth arc hole pair i on the top plate 34 are symmetrically located on the left and right sides of the tetragonal pyramid 38. The second ball cover group 33 includes four ball covers, which are respectively fixedly connected on four flanges of the second flange group 37. The second ball rod group 32 includes four ball rods, each of which is formed by fixedly connecting the second ball body 40 and the second threaded rod 39. The second spring seat group 31 includes four spring seats, and a center of each spring seat is provided with a second threaded hole k. Two ends of the transverse plate 36 are connected with two arc holes of the fourth arc hole pair i of the top plate 34 through bolts. Four second ball bodies 40 of the second ball rod group 32 are movably connected with four stepped holes of the second stepped hole group j of the top plate 34. Four second threaded holes k of the second spring seat group 31 are threaded with four second threaded rods 39 of the second ball rod group 32.

The angle adjustment process of the joint axis of some embodiments in the present disclosure is as follows:

1. unscrewing the bolt 20 connecting the subtalar joint axis 16 and the ankle axis 18;

2. unscrewing the bolt connecting the second arc plate assembly 30 and the top plate 34, unscrewing the bolt connecting the fifth arc hole pair g and the third perforated block 29a of the left connecting block 17a, and unscrewing the bolt connecting the sixth arc hole pair h and the fourth perforated block 29b of the right connecting block 17b.

3. rotating the second arc plate assembly 30 to an ideal position along the fourth arc hole pair i, and screwing the bolt connecting the second arc plate assembly 30 and the top plate 34;

4. rotating the ankle axis 18 to an ideal position along the fifth arc hole pair g, screwing the bolt connecting the fifth arc hole pair g and the third perforated block 29a of the left connecting block 17a, and screwing the bolt connecting the sixth arc hole pair h and the fourth perforated block 29b of the right connecting block 17b;

5. unscrewing the bolt connecting the first arc plate assembly 9 and the base plate 5, unscrewing the bolt connecting the second arc hole pair e and the first perforated block 25a of the front connecting block 19a, and unscrewing the bolt connecting the third arc hole pair f and the second perforated block 25b of the rear connecting block 19b;

6. rotating the first arc plate assembly 9 to an ideal position along the first arc hole pair b, and screwing the bolt connecting the first arc plate assembly 9 and the base plate 5;

7. rotating the subtalar joint axis 16 to an ideal position along the second arc hole pair e, screwing the bolt connecting the second arc hole pair e and the first perforated block 25a of the front connecting block 19a, and screwing the bolt connecting the third arc hole pair f and the second perforated block 25b of the rear connecting block 19b; and

8. screwing the bolt 20 connecting the subtalar joint axis 16 and the ankle axis 18.

What is claimed is:

1. A small-size multi-axis ankle prosthesis, comprising a lower part (A), a joint axis (B), an upper part (C), a first spring (1), a second spring (2), a third spring (3) and a fourth spring (4),
wherein the lower part (A) comprises a base plate (5), a first ball cover group (6), a first ball rod group (7), a first spring seat group (8) and a first arc plate assembly (9); the base plate (5) is disc-shaped, and an outer periphery of the base plate is uniformly provided with four flanges of a first flange group (10);
the first arc plate assembly (9) comprises a first arc plate (15) and a transverse plate (14), and the first arc plate (15) is fixed on an upper surface of the transverse plate (14); two arc holes of a second arc hole pair (e) are provided on the first arc plate (15) adjacent to a front end thereof; two arc holes of a third arc hole pair (f) are provided on the first arc plate (15) adjacent to a rear end thereof;
the joint axis (B) comprises a subtalar joint axis (16), a left connecting block (17a), a right connecting block (17b), an ankle axis (18), a front connecting block (19a), a rear connecting block (19b), a bolt (20), and an oil-free bushing group (21); wherein the left connecting block (17a) comprises a first cylindrical cap (24a) and a first perforated block (25a) which is fixed at a center portion on a front end of the first cylindrical cap (24a); the right connecting block (17b) comprises a second cylindrical cap (24b) and a second perforated block (25b) which is fixed at a center portion on a rear end of the second cylindrical cap (24b); the front connecting block (19a) comprises a third cylindrical cap (28a) and a third perforated block (29a) which is fixed at a center portion on a left end of the third cylindrical cap (28a); the rear connecting block (19b) comprises a fourth cylindrical cap (28b) and a fourth perforated block (29b) which is fixed at a center portion on a right end of the fourth cylindrical cap (28b);
the upper part (C) comprises a second arc plate assembly (30), a second spring seat group (31), a second ball rod group (32), a second ball cover group (33) and a top plate (34), the top plate (34) is disc-shaped and an outer periphery of the top plate is uniformly provided with four flanges of a second flange group (37);
the second arc plate assembly (30) comprises a second arc plate (35) and a transverse plate (36); the second arc plate (35) is fixedly connected to a bottom of the transverse plate (36), a fifth arc hole pair (g) is provided on the second arc plate (35) adjacent to a left lower end thereof, and a sixth arc hole pair (h) is provided on the second arc plate (35) adjacent to a right lower end thereof;
the four flanges of the second flange group (37) of the top plate (34) in the upper part (C) are symmetrical with the four flanges of the first flange group (10) of the base plate (5) in the lower part (A) in an up-down direction; upper ends of the first spring (1), the second spring (2), the third spring (3) and the fourth spring (4) are respectively fixed with four spring seats of the second spring seat group (31) in the upper part (C); lower ends of the first spring (1), the second spring (2), the third spring (3) and the fourth spring (4) are respectively fixedly connected with four spring seats of the first spring seat group (8) in the lower part (A);
the joint axis (B) is located in a space surrounded by the lower part (A), the first spring (1), the second spring (2), the third spring (3), the fourth spring (4) and the upper part (C); the third perforated block (29a) of the front connecting block (19a) in the joint axis (B) is connected with the fifth arc hole pair (g) of the second arc plate (35) in the upper part (C) through a bolt; the fourth perforated block (29b) of the rear connecting block (19b) in the joint axis (B) is connected with the sixth arc hole pair (h) of the second arc plate (35) in the upper part (C) through a bolt; the first perforated block (25a) of the left connecting block (17a) in the joint axis (B) is connected with the second arc hole pair (e) of the first arc plate (15) in the lower part (A) through a bolt; the second perforated block (25b) of the right connecting block (17b) in the joint axis (B) is connected with the third arc hole pair (f) of the first arc plate (15) in the lower part (A) through a bolt.

2. The small-size multi-axis ankle prosthesis according to claim 1, wherein the four flanges of the first flange group (10) of the base plate (5) in the lower part (A) are provided with four holes of the first stepped hole group (a) respectively, a back surface of the base plate (5) is provided with a boss (11), and a center portion of the base plate 5 is provided with a first threaded hole (c), two arc holes of the first arc hole pair (b) provided in the base plate (5) are symmetrically located on a front side and a rear side of the boss (11) respectively; the first ball cover group (6) comprises four ball covers; the first ball rod group (7) comprises four ball rods, each of the ball rods is formed by fixedly connecting a first ball body (12) and a first threaded rod (13); the first spring seat group (8) comprises four spring seats, and a center of each spring seat is provided with a second threaded hole (d); two ends of the transverse plate (14) are connected with the two arc holes of the first arc hole pair (b) in the base plate (5) through bolts; the four first ball bodies (12) of the first ball rod group (7) are movably connected with four stepped holes of the first stepped hole group (a) in the base plate (5); four ball covers of the first ball cover group (6) are respectively fixed under the four flanges of the first flange group (10); the four second threaded holes (d) of the first spring seat group (8) are in threaded connection with the four first threaded rods (13) of the first ball rod group (7).

3. The small-size multi-axis ankle prosthesis according to claim 1, the oil-free bushing group (21) in the joint axis (B) comprises four oil-free bushings; the subtalar joint axis (16) comprises a first round plate (22) with a threaded hole, and a first cylinder pair (23); two cylinders of the first cylinder pair (23) are respectively fixedly connected to a front side and a rear side of the first round plate (22) with the threaded hole; the ankle axis (18) comprises a second round plate (26) with a threaded hole, and a second cylinder pair (27); and two cylinders of the second cylinder pair (27) are fixedly connected to a left side and a right side of the second round plate (26) with the threaded hole respectively; the ankle axis (18) and the subtalar joint axis (16) are arranged with one on another and in a cross mode, and are fixed together by the bolt (20); the oil-free bushing group (21) comprises four oil-free bushings; two connecting blocks of the first connecting block pair (17) are movably connected with the two cylinders of the second cylinder pair (27) in the ankle axis (18) through left and right oil-free bushings of the oil-free bushing group (21); two connecting blocks of the second connecting block pair (19) are movably connected with the two cylinders of the first cylinder pair (23) in the subtalar joint axis (16) through front and rear oil-free bushings of the oil-free bushing group (21).

4. The small-size multi-axis ankle prosthesis according to claim 1, wherein the four flanges of the second flange group (37) of the top plate (34) in the upper part (C) are provided with four stepped holes of the second stepped hole group (j) respectively; a tetragonal pyramid (38) is fixedly provided on a center of the top plate (34); two arc holes of the fourth arc hole pair (i) in the top plate (34) are symmetrically located on left and right sides of the tetragonal pyramid (38); the second ball cover group (33) comprises four ball covers, which are respectively fixed on the four flanges of the second flange group (37); the second ball rod group (32) includes four ball rods, each ball rod is formed by fixedly connecting the second ball body (40) and the second threaded rod (39); the second spring seat group (31) comprises four spring seats, and a center of each spring seat is provided with a second threaded hole (k); two ends of the transverse plate (36) are connected with the two arc holes of the fourth arc hole pair (i) of the top plate (34) through bolts; four second ball bodies (40) of the second ball rod group (32) are movably connected with four stepped holes of the second stepped hole group (j) of the top plate (34); four second threaded holes (k) of the second spring seat group (31) are in threaded connection with four second threaded rods (39) of the second ball rod group (32).
